(19) United States
(12) Patent Application Publication
(10) Pub. No.: US 2020/0383816 A1
(43) Pub. Date: Dec. 10, 2020

(54) HEAD AND NECK SUPPORT UNIT

(71) Applicant: ASMED OZEL SAC EKIMI SAGLIK HIZMETLERI TICARE LIMIED SIRKET, Atasehie (TR)

(72) Inventor: Koray ERDOGAN, Kadikoy (TR)

(21) Appl. No.: 16/770,128

(22) PCT Filed: Dec. 5, 2018
(86) PCT No.: PCT/TR2018/000050

§ 371 Date: Jun. 5, 2020

(30) Foreign Application Priority Data
Dec. 5, 2017 (TR) 2017/19598

(51) Int. Cl. A61F 5/055; A61H 1/00; A61G 13/12

(52) U.S. Cl. CPC A61F 5/055; A61H 1/00; A61H 2205/02; A61H 2205/04; A61G 13/121

(57) ABSTRACT

The invention relates to a unit used in the medical field in order to keep head and neck stationary and support them in the operations performed in head area while the patient is in the sitting position. The invention may be used as a supporting means in different operation and treatments requiring immobilization and a sitting position in which the center of gravity of the patient is in front or in perpendicular axis. The invention functions as a bridge transmitting the whole load to the leg area by supporting spine and neck area under the chin and neck. It ensures the health, comfort and safety the spine by removing the load on the neck muscles and cervical vertebrae during the operation. It helps the operator and medical staff to work more safely and in a more efficient way by means of immobilization.

### TECHNICAL FIELD

The invention relates to a unit used in the medical field in order to keep head and neck stationary and support them in the operations performed in head area while the patient is in the sitting position. The invention may be used as a supporting means in different operation and treatments requiring immobilization and a sitting position in which the center of gravity of the patient is in front or in perpendicular axis.

### STATE OF THE ART

In the operations performed in the head area in medical fields, immobilizing the head of the patient generally ensured by the operator and assistant staff. Cushioning or cervical collar type immobilization solutions surrounding the head are also used for critical situations. This restricts the operation area; however it does not decrease the load on the spine.

Moreover, there are many types of cervical collars in the market which are already used in neck injuries, pain situations and neck stiffness.

### Advantages of the Invention

The invention functions as a bridge transmitting the whole load to the leg area by supporting spine and neck area under the chin and neck. It ensures the health, comfort and safety the spine by removing the load on the neck muscles and cervical vertebrae during the operation. It helps the operator and medical staff to work more safely and in a more efficient way by means of immobilization.

The invention is useful to immobilize the head and to decrease the load on the neck area during the operations performed in the head area in the sitting position. It is not a unit which is located around the head and immobilizing the head mechanically. It forms a support in the chin and cheek areas in the natural posture of the person so as to keep the posture. It creates a bridge along the spine by transmitting the load to the legs.

The invention has a feature to enclose the chest area and to be adjusted in the control of the patient and according to the people having different physical structures. It is easily located thanks to its light and portable feature. It inhibits neck and back muscles to be tired in long-term operations. It eliminates negative effects on the patient by protecting the posture and correct spine position. It enhances working comfort of the operators performing critical operation on the patient.

### DESCRIPTION OF THE INVENTION

Present invention relates to a head and neck support unit developed in order to prevent the disadvantages mentioned above and to supply the technical field with new advantages.

### DRAWINGS

The applications of the present invention summarized briefly above and addressed in more detail below may be understood by referring to the exemplary applications defined in the appended drawings of the invention. However, it has to be mentioned that the appended drawings illustrates the typical applications of this invention only, thus it cannot be assumed that they limit the scope of the invention, because it may allow for equally effective applications.

For the ease of understanding, identical reference numbers are used in the possible cases in order to define the identical members common in the figures. The figures have not been drawn to scale and they may be simplified for clarity. It has to be considered that the members and properties of an application may be included in the other applications usefully without any need for further explanation.

### DESCRIPTION OF THE DETAILS IN DRAWINGS

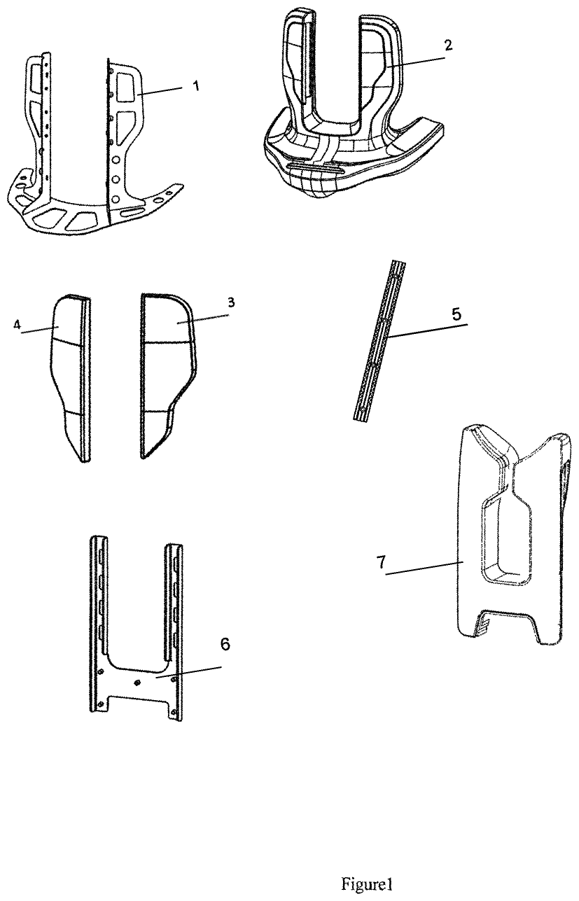

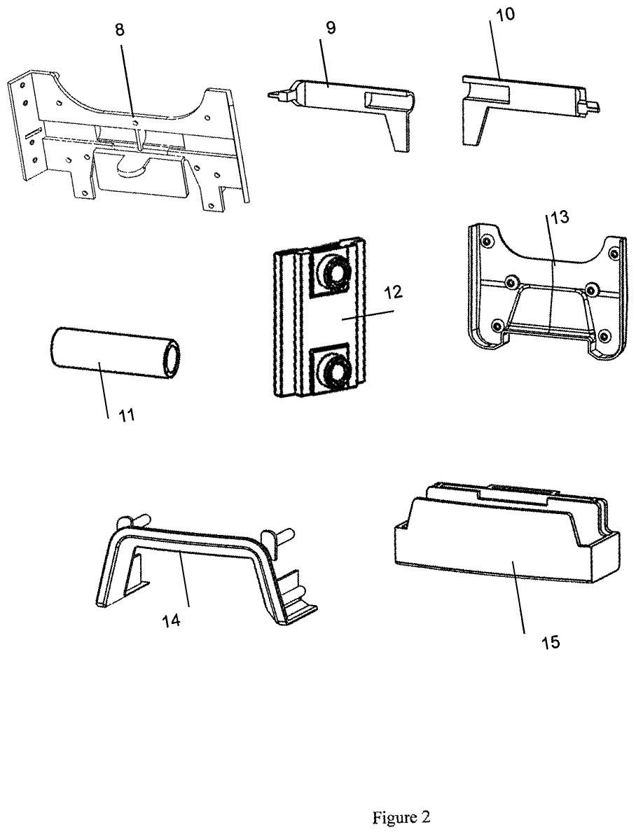

FIG. 1 and FIG. 2 is the general view of the all parts constituting the invention.

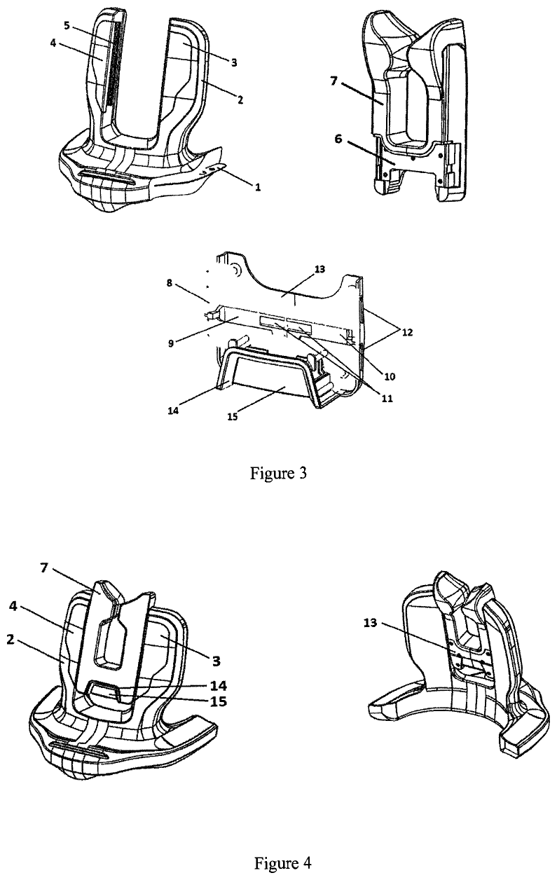

FIG. 3 is the general view of body, cushion and locking sections of the constituent parts of the invention.

FIG. 4 is front and back perspective views of the invention.

The equivalences of the reference numbers used in the figures are presented below:
1—Bracket of the body,
2—Polyurethane body,
3—Right decorative part of the body,
4—Left decorative part of the body,
5—Slide,
6—Bracket of the cushion,
7—Polyurethane cushion,
8—Body of the mechanism,
9—Left lock,
10—Right lock,
11—Spring,
12—Slide carriage,
13—Mechanism cover,
14—Button Frame,
15—Button.

### DETAILED DESCRIPTION OF THE INVENTION

In this detailed description, preferred alternatives of the head and neck support embodiments of the invention are described for better understanding the subject and without any limiting effect.

The invention may be used as a supporting means in different operation and treatments requiring immobilization and a sitting position in which the center of gravity of the patient is in front or in perpendicular axis.

Bracket of the body (1) is manufactured integrally by locating within the polyurethane body (2) during manufacturing. Right decorative part (3) and left decorative part (4) of the body are adhesively attached to the polyurethane body (2) and slide (5) is screwed and fixed into the bracket of the body (1). Bracket of the cushion (6) is manufactured integrally being located within polyurethane cushion (7). Left lock (9), right lock (10) and spring (11) parts are attached to the mechanism body (8). Spring part (11) applies a driving power so as to move lock parts (9, 10) from each other. Slide carriage (12) is fixed to the mechanism body (8) by screwing and grouped mechanism body (8) is screwed to the polyurethane cushion (7). Button (15) is attached to the mechanism body (8) by means of button frame (14). Mechanism cover (13) is screwed to the mechanism body (8). Body group consists of 1-2-3-4-5 numbered parts and cushion group consists of 6-7-8-9-10-11-12-13-14-15 numbered parts. Upon grouping these parts according to the description, slide carriage parts (12) are attached to the slide (5) part.

The functions of the parts of the invention are as follows:

Bracket of the body (1) is used in order to provide strength to the polyurethane body (2).

Polyurethane body (2) forms the body of the invention.

Left and right decorative parts of the body (4, 3) are the parts which are used for improve the appearance.

The slide (5) is fixed by attaching it to the bracket of the body (1), and parts of the slide carriage (12) moves on the slide (5) part and enable cushion group to move in one axis.

Bracket of the cushion (6) is used in order to provide strength to the polyurethane cushion (7).

Polyurethane cushion moves independently from the polyurethane body (2) and it enables the length of the product to be longer and shorter.

Mechanism body (8) keeps slide carriage (12), left lock (9), right lock (10), and spring (11) stationary.

Left lock (9) and right lock (10) are attached to the notches on the slide (5) and it fixes the mobile part of the product in any location.

The spring (11) enables locking by pushing the left lock (9) and right lock (10).

Slide carriage (12) provides movement on the slide (5).

Mechanism cover (13) protects the mechanism section.

Button frame (14) bears the button part (15).

Button (15) enables right and left locks (9, 10) to be open by pulling them backward.

Manufacturing of the invention comprises following steps:
1. The supporting brackets (1, 6) are manufactured. These parts are formed by laser cutting and boiling the sheet metal material. Aluminum is preferred so as to enable the components not to be heavy.
2. The bodies (2, 7) are manufactured. In this step, metal brackets (1, 6) which have been manufactured in the previous step are placed in the polyurethane mold created in accordance with the design and body (2) and cushion (7) parts are manufactured by injecting suitable type polyurethane into the mold.
3. Plastic components are manufactured. 3 dimensional printers/silicone molding or plastic injection methods are used for manufacturing these components consisting of the mechanical parts of the product. These parts are manufactured from ABS or equivalent material in accordance with the engineering drawing carried out by one of these 3 processes.
4. Semi-finished parts are gathered together. In this step, polyurethane bodies (2, 7) and plastic mechanical parts (3, 4, 8, 9, 10, 11, 12, 13, 14, and 15) are mounted by means of suitable assembly methods.

The invention may respond to much more physical variables in different sizes and different forms in terms of structure and manufacturing way. Alternatively, sitting position may be supplied in anesthetized patients by forming a fixing option on the human body by means of a part to be attached to the body of the invention. Furthermore, the invention may be used as a supporting means in different operation and treatments requiring immobilization and a sitting position in which the center of gravity of the patient is in front or in perpendicular axis.

1. A Neck and Head Support Unit consisting of:
bracket of the body (1),
polyurethane body (2),
right decorative part of the body (3),
left decorative part of the body (4),
slide (5),
bracket of the cushion (6),
polyurethane cushion (7),
mechanism body (8),
left lock (9),
right lock (10),
spring (11),
slide carriage (12),
mechanism cover (13),
button frame (14), and
button (15).

2. Neck and Head Support Unit according to claim 1 characterized in that bracket of the body (1) is manufactured integrally by locating within the polyurethane body (2) during manufacturing, right decorative part (3) and left decorative part (4) of the body are adhesively attached to the polyurethane body (2), the slide (5) is screwed and fixed into the bracket of the body (1), bracket of the cushion (6) is manufactured integrally being located within polyurethane cushion (7), left lock (9), right lock (10) and spring (11) parts are attached to the mechanism body (8), spring part (11) applies a driving power so as to move lock parts (9, 10) from each other, slide carriage (12) is fixed to the mechanism body (8) by screwing, grouped mechanism body (8) is screwed to the polyurethane cushion (7), button (15) is attached to the mechanism body (8) part by means of button frame (14) and mechanism cover (13) is screwed to the mechanism body (8).

3. Neck and Head Support Unit according to claim 1 characterized in that polyurethane cushion (7) moves independently from polyurethane body (2) and enables the product length to become longer and shorter.
